(19) United States
(12) Patent Application Publication
(10) Pub. No.: US 2007/0203331 A1
(43) Pub. Date: Aug. 30, 2007

(54) Dna enzyme and method for controlling activity thereof

(75) Inventors: Hiroyuki Asanuma, Saitama (JP); Makoto Komiyama, Tokyo (JP); Daijiro Matsunaga, Tokyo (JP); Takeshi Kuramochi, Tokyo (JP)

Correspondence Address: BIRCH STEWART KOLASCH & BIRCH, PO BOX 747, FALLS CHURCH, VA, 22040-0747, US

(73) Assignee: JAPAN SCIENCE AND TECHNOLOGY AGENCY, KAWAGUCHI-SHI (JP)

(21) Appl. No.: 10/590,777

(22) PCT Filed: Feb. 24, 2005
(86) PCT No.: PCT/JP05/03052

§ 371 Date: Jan. 4, 2007

(30) Foreign Application Priority Data
Feb. 27, 2004 (JP) 2004-055086

(51) Int. Cl. C07H 21/04

(52) U.S. Cl. 534/727; 536/23.2

(57) ABSTRACT

A DNA enzyme having the RNA cleavage activity significantly improved as compared with those of known DNA enzymes and a method for controlling the activity, the method being capable of reversibly controlling the RNA cleavage activity of the DNA enzyme by light irradiation, are provided. The DNA enzyme includes a nucleotide residue, to which any one organic group selected from the group consisting of azobenzene, spiropyran, stilbene, and derivatives thereof is bonded, at a 3′-side end of a catalytically active loop of the DNA enzyme. The method for controlling the activity includes the step of applying light at specific wavelengths to the DNA enzyme including a nucleotide residue, to which any one organic group selected from the group consisting of azobenzene, spiropyran, stilbene, and derivatives thereof is bonded, and thereby, effecting reversible structural isomerization between a planar structure and a nonplanar structure of the organic group, so as to control the RNA cleavage activity of the DNA enzyme.

### TECHNICAL FIELD

The present invention relates to a DNA enzyme and a method for controlling the activity thereof. In particular, it relates to a DNA enzyme having the RNA cleavage activity significantly improved as compared with that of a DNA enzyme simply composed of four natural bases and a method for controlling the activity of a DNA enzyme by light irradiation at specific wavelengths.

### Background Art

If an RNA can be selectively hydrolyzed on a sequence basis, gene expression at a messenger RNA level can be suppressed, and an application to the therapy for diseases based on genes can be expected. Naturally occurring RNase is not a protein. It is simply composed of an RNA, and is referred to as ribozyme. However, the RNA is unstable and tends to be decomposed. Therefore, a more stable DNA hydrolytic enzyme (artificial enzyme) has been required. In response to the requirement, Joyce et al., in the U.S., have proposed an RNA hydrolytic enzyme simply composed of a naturally occurring DNA in 1997 for the first time in the world (Non-patent Document 1).

The RNA hydrolytic enzyme simply composed of a DNA is generally referred to as a DNA enzyme (deoxyribozyme, DNAzyme), and is an artificial ribonuclease developed by an in vitro selection method. Since an in vivo metal, Mg²⁺, serves as a cofactor, an in vivo application is possible. Specific contents thereof are disclosed in Non-patent Document 1. An 8-17 DNA enzyme and 10-23 DNA enzyme are included, and sequence formulae thereof are as described below.

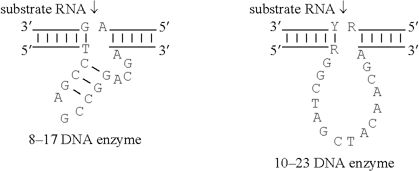

In the above-described sequence formulae, arrows indicate cleavage sites. The base sequence of the substrate RNA at the cleavage site is GA for the 8-17 DNA enzyme, and is Y(U or C)R(A or G) for the 10-23 DNA enzyme. The sequence of the DNA enzyme becomes a sequence complementary to the substrate RNA. However, CCGAGCCGGACGA (sequence number 1) in the 8-17 DNA enzyme and GGCTAGCTACAACGA (sequence number 2) in the 10-23 DNA enzyme are catalystically active loops, and are not complementary to the substrate RNA.

On the other hand, Non-patent Document 2 reports the gene expression control by the light irradiation, and the gene expression control is conducted by using an artificial DNA in which azobenzene has been introduced in a side chain of the DNA. Specifically, since reversible structural isomerization between a trans form (planar structure) and a cis form (nonplanar structure) of azobenzene is effected by light irradiation at specific wavelengths, it becomes possible to, for example, optically control the formation and dissociation of a duplex of the DNA and optically control the formation of a triplex by taking advantage of this characteristic of azobenzene.
Non-patent Document 1: Proceedings of the National Academy of Science of the United States of America 94. 4262-4266 (1997)
Non-patent Document 2: Journal of Japanese Society for Biomaterials 21. 290-296 (2003)

### DISCLOSURE OF INVENTION

For the DNA enzyme shown in Non-Patent Document 1, the RNA cleavage activity itself is not high, and is very low as compared with that of natural ribozyme. Consequently, the DNA enzyme has been required to have higher activity.

It is believed that the control of the RNA cleavage activity of the DNA enzyme is very difficult. Therefore, if the activity can be reversibly controlled by an external stimulus, e.g., light irradiation, without changing the condition in the reaction system, the usefulness thereof can be increased significantly.

Accordingly, it is an object of the present invention to provide a DNA enzyme having the RNA cleavage activity significantly improved as compared with those of known DNA enzymes.

It is another object of the present invention to provide a method for controlling the activity, the method being capable of reversibly controlling the RNA cleavage activity of the DNA enzyme by light irradiation.

### Means for Solving the Problems

In order to overcome the above-described problems, the inventors of the present invention have conducted intensive research. As a result, it has been found that the above-described object can be achieved by introducing a nucleotide residue having a planar structure at a predetermined site of a DNA enzyme, so that the DNA enzyme of an aspect of the present invention has been completed.

That is, the DNA enzyme of an aspect of the present invention is characterized by including a nucleotide residue, to which any one organic group selected from the group consisting of azobenzene, spiropyran, stilbene, and derivatives thereof is bonded, at a 3′-side end of a catalytically active loop of the DNA enzyme.

The inventors of the present invention have found that reversible structural isomerization from the above-described planar structure to a nonplanar structure has been able to be effected by applying the light at a specific wavelength to the above-described DNA enzyme, the RNA cleavage activity of the DNA enzyme have been able to be controlled, and the above-described other object has been able to be achieved, so that the method for controlling the activity according to an aspect of the present invention has been completed.

That is, the method for controlling the activity according to an aspect of the present invention is characterized by including the step of applying light at specific wavelengths to the DNA enzyme including a nucleotide residue, to which any one organic group selected from the group consisting of azobenzene, spiropyran, stilbene, and derivatives thereof is bonded, and thereby, effecting reversible structural isomerization between a planar structure and a nonplanar structure of the organic group, so as to control the RNA cleavage activity of the DNA enzyme.

### Advantages

For the DNA enzyme including a nucleotide residue having a planar structure of the present invention, the RNA cleavage activity is significantly improved as compared with that of a DNA enzyme simply composed of four natural bases. According to the method for controlling the activity of a DNA enzyme of the present invention, it becomes possible to reversibly control the cleavage activity of the DNA enzyme by light irradiation at specific wavelengths, and it can be expected that in vivo gene expression is optically controlled.

### BEST MODE FOR CARRYING OUT THE INVENTION

The preferable embodiments of the present invention will be specifically described below.

The DNA enzyme according to the present invention is a chemically modified DNA enzyme, wherein a nucleotide residue, to which any one organic group selected from the group consisting of azobenzene, spiropyran, stilbene, and derivatives thereof is bonded, has been introduced at a 3′-side end of a catalytically active loop of the DNA enzyme disclosed in the above-described Non-Patent Document 1. The base sequence of the above-described DNA enzyme except the catalytically active loop is a base complementary to the substrate RNA. However, the base sequence of the RNA enzyme is not specifically limited.

The DNA enzyme according to the present invention is represented by, for example, the following Formula. 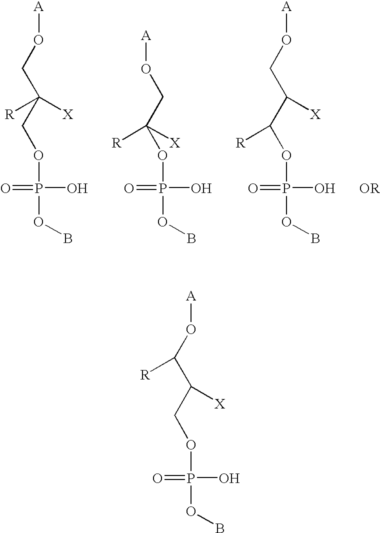
In the above-described Formulae, A represents a catalytically active loop end, B represents nucleotide or oligonucleotide. X represents any one organic group selected from the group consisting of azobenzene, spiropyran, stilbene, and derivatives thereof. R represents an unsubstituted or a halogen atom-, hydroxyl-, amino-, nitro-, or carboxyl-substituted alkyl group or alkoxy group having the carbon number of 1 to 20, preferably of 1 to 10, and more preferably of 1 to 4; an unsubstituted or a halogen atom-, hydroxyl-, amino-, nitro-, or carboxyl-substituted alkenyl group or alkynyl group having the carbon number of 2 to 20, preferably of 2 to 10, and more preferably of 2 to 4; a hydroxyl group; a halogen atom; an amino group; a nitro group; or a carboxyl group.

Preferably, the above-described X is azobenzene or a derivative thereof. Any group may be intercalated in a portion bonded to the nucleotide residue. Examples of X can include organic groups represented by the following Formula (I), (II), or (III).

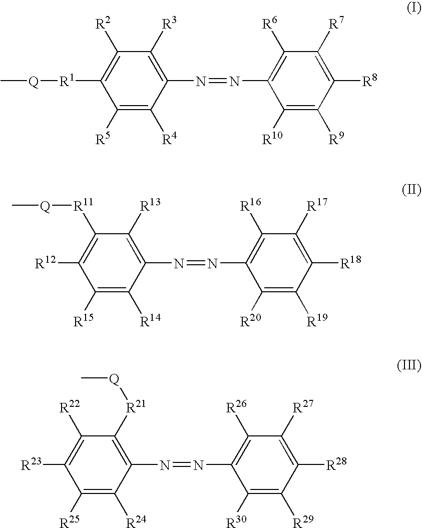

In the above-described Formulae (I), (II), and (III), R¹, R¹¹, and R²¹ represent independently a direct bond; an unsubstituted or a halogen atom-, hydroxyl-, amino-, nitro-, or carboxyl-substituted alkylene group having the carbon number of 1 to 20, preferably of 1 to 10, and further preferably of 1 to 4; or an unsubstituted or a halogen atom-, hydroxyl-, amino-, nitro-, or carboxyl-substituted alkenylene group having the carbon number of 2 to 20, preferably of 2 to 10, and further preferably of 2 to 4. Q represents a direct bond, an oxygen atom, a —(CH₂)ₙ—NH—CO— group, or a —(CH₂)ₙ—CO—NH— group, where n=1 to 5. R² to R¹⁰, R¹² to R²⁰, and R²² to R³⁰ represent independently an unsubstituted or a halogen atom-, hydroxyl-, amino-, nitro-, or carboxyl-substituted alkyl group or alkoxy group having the carbon number of 1 to 20, preferably of 1 to 10, and further preferably of 1 to 4; an unsubstituted or a halogen atom-, hydroxyl-, amino-, nitro-, or carboxyl-substituted alkenyl group or alkynyl group having the carbon number of 2 to 20, preferably of 2 to 10, and further preferably of 2 to 4; a hydroxyl group; a halogen atom; an amino group; a nitro group; or a carboxyl group.

The synthesis of the DNA enzyme including the nucleotide residue, according to the present invention, can be conducted in accordance with known techniques, e.g., techniques described in The Journal of Organic Chemistry 62. 846-852 (1997), Tetrahedron Letters 39. 9019-9022 (1998), and Angewandte Chemie International edition 40. 2671-2673 (2001). Phosphoamidite monomers corresponding to individual nucleotide residues are synthesized, a known DNA synthesizer is used and, thereby, DNA enzymes including desired nucleotide residues can be synthesized. In this case, polymethylene chains having various lengths can be used. However, an unsubstituted or an alkyl-substituted ethylene chain or a trimethylene chain is preferable. In this case, preferably, an organic group to be introduced is introduced as if to form a covalent bond to any one of carbon atoms for the ethylene chain, or to a central carbon atom for the trimethylene chain.

A method for controlling the RNA cleavage activity of the DNA enzyme will be described below. A DNA enzyme including a nucleotide residue, to which any one organic group selected from the group consisting of azobenzene, spiropyran, stilbene, and derivatives thereof is bonded, is used and is irradiated with light at specific wavelengths, wherein structural isomerization between a planar structure and a nonplanar structure of the organic group is effected by the light irradiation at specific wavelengths. Consequently, reversible structural isomerization between a planar structure and a nonplanar structure of the above-described organic group is effected and it becomes possible to control the RNA cleavage activity. Here, the base sequence of the DNA enzyme except the catalytically active loop is a base complementary to the substrate RNA. However, the base sequence of the RNA is not specifically limited.

When the introduction position of the above-described nucleotide residue is the 3′-side end of the catalytically active loop, the DNA enzyme according to the present invention is derived, and a high RNA cleavage activity is exhibited. However, in the method for controlling the activity according to the present invention, the above-described introduction position is not specifically limited, and may be in oligonucleotide complementary to the substrate RNA. Such a DNA enzyme is represented by the following Formula, for example.

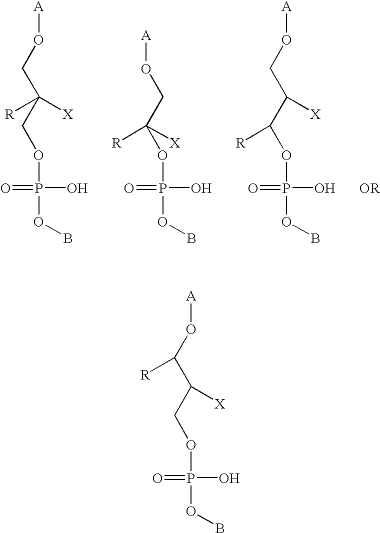

In the above-described Formulae, A and B represent independently a hydrogen atom, nucleotide or oligonucleotide. However, A and B do not represent a hydrogen atom at the same time. X represents any one organic group selected from the group consisting of azobenzene, spiropyran, stilbene, and derivatives thereof. R represents an unsubstituted or a halogen atom-, hydroxyl-, amino-, nitro-, or carboxyl-substituted alkyl group or alkoxy group having the carbon number of 1 to 20, preferably of 1 to 10, and more preferably of 1 to 4; an unsubstituted or a halogen atom-, hydroxyl-, amino-, nitro-, or carboxyl-substituted alkenyl group or alkynyl group having the carbon number of 2 to 20, preferably of 2 to 10, and more preferably of 2 to 4; a hydroxyl group; a halogen atom; an amino group; a nitro group; or a carboxyl group.

Preferably, the above-described X is azobenzene or a derivative thereof. In this case, the benzene ring may have any substituent as long as the function of controlling the enzyme activity through reversible structural isomerization by the light irradiation is not impaired, and any group may be intercalated in a portion bonded to the nucleotide residue. Preferably, a substituent and an intercalating group at a para position of azobenzene are groups which do not take on a resonance structure with the benzene ring.

This is because a substituent, e.g., a carboxyl group, an amino group, or a nitro group, at a para position and an amide bond at a para position take on a resonance structure at the para position and, thereby, isomerization between a cis form (nonplanar structure) and a trans form (planar structure) of azobenzene tends to be effected thermally. Preferably, a substituent at a meta position is a group other than the nitro group. Examples of X can include organic groups represented by the following Formula (IV), (V), or (VI).

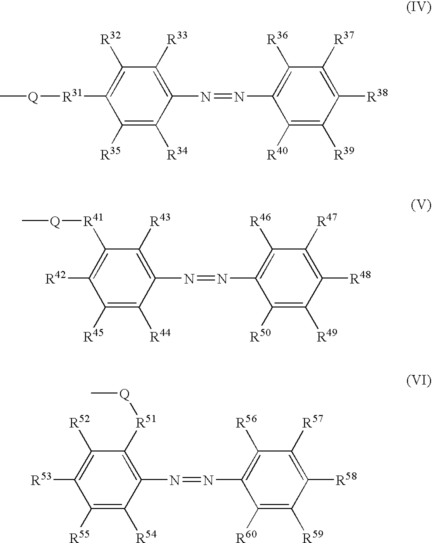

In the above-described Formulae (IV), (V), and (VI), R³¹, R⁴¹, and R⁵¹ represent independently a direct bond; an unsubstituted or a halogen atom-, hydroxyl-, amino-, nitro-, or carboxyl-substituted alkylene group having the carbon number of 1 to 20, preferably of 1 to 10, and further preferably of 1 to 4; or an unsubstituted or a halogen atom-, hydroxyl-, amino-, nitro-, or carboxyl-substituted alkenylene group having the carbon number of 2 to 20, preferably of 2 to 10, and further preferably of 2 to 4. Q represents a direct bond, an oxygen atom, a —(CH₂)ₙ—NH—CO— group, or a —(CH₂)ₙ—CO—NH— group, where n=1 to 5. R³² to R³⁷, R³⁹, R⁴⁰, R⁴² to R⁴⁷, R⁴⁹, R⁵⁰, R⁵² to R⁵⁷, R⁵⁹, and R⁶⁰ represent independently an unsubstituted or a halogen atom-, hydroxyl-, amino-, nitro-, or carboxyl-substituted alkyl group or alkoxy group having the carbon number of 1 to 20, preferably of 1 to 10, and further preferably of 1 to 4; an unsubstituted or a halogen atom-, hydroxyl-, amino-, nitro-, or carboxyl-substituted alkenyl group or alkynyl group having the carbon number of 2 to 20, preferably of 2 to 10, and further preferably of 2 to 4; a hydroxyl group; a halogen atom; an amino group; a nitro group; or a carboxyl group. R³⁸, R⁴⁸, and R⁵⁸ represent independently an unsubstituted or a halogen atom-, hydroxyl-, amino-, nitro-, or carboxyl-substituted alkyl group or alkoxy group having the carbon number of 1 to 20, preferably of 1 to 10, and further preferably of 1 to 4; an unsubstituted or a halogen atom-, hydroxyl-, amino-, nitro-, or carboxyl-substituted alkenyl group or alkynyl group having the carbon number of 2 to 20, preferably of 2 to 10, and further preferably of 2 to 4; a hydroxyl group; or a halogen atom. Preferably, in Formula (IV), -Q-R³¹— is an intercalating group which does not take on a resonance structure with azobenzene.

Every light with a wavelength within the range from the ultraviolet region to the infrared region can be used as the light applied to effect the structural isomerization of the above-described organic group, as long as the light can isomerizes the organic group. However, 300 nm or more is preferable because the DNA is not damaged. For example, the structural isomerization from one isomer to the other isomer can be effected by applying the light (UV light) of 300 to 400 nm, and a reverse change can be effected by applying the light (visible light) of 400 nm or more.

### EXAMPLES

The present invention will be described below in further detail with reference to examples. However the present invention is not limited to these examples.

### Synthesis Example 1

“Synthesis of DNA Enzyme Including Azobenzene Derivative”

The synthesis was conducted on the basis of the following scheme.

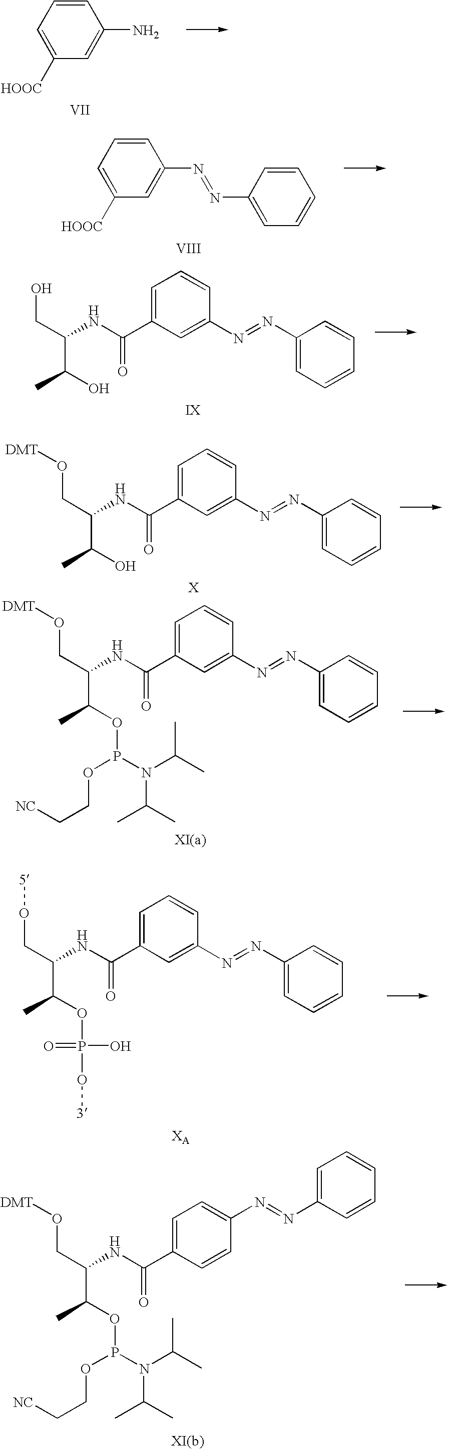

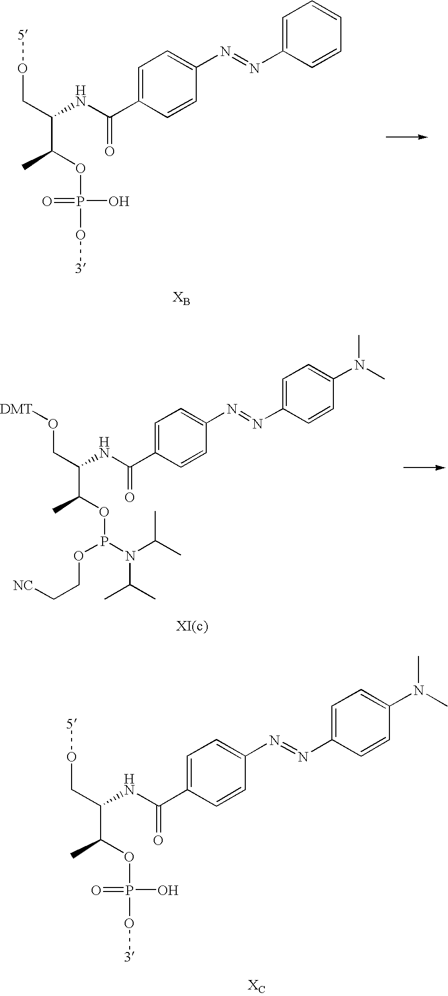

An unrefined product of 3-phenylazobenzoic acid VIII was produced by dissolving 3-aminobenzoic acid VII into acetic acid, mixing an acetic acid solution of nitrosobenzene therewith, and agitating for 12 hours at room temperature. The resulting unrefined product was refined through recrystallization by using ethanol. The resulting 3-phenylazobenzoic acid VIII and D-threoninol were reacted in N,N-dimethylformamide (DMF) in the presence of dicyclohexylcarbodiimide and 1-hydroxybenzotriazol, so that an unrefined product of Compound IX in which 3-phenylazobenzoic acid VIII and D-threoninol were bonded by an amide bond.

The resulting Compound IX was separated and refined by column chromatography and, thereafter, was reacted with 4,4′-dimethoxytrityl chloride in a pyridine-dichloromethane mixed solvent in the presence of 4-dimethylaminopyridine on the basis of the technique described in Angewandte Chemie International edition 40. 2671-2673 (2001), so that an unrefined product of Compound X, in which one hydroxyl group is protected by the 4,4′-dimethoxytrityl (DMT) group, was produced. The resulting Compound X was separated and refined by the column chromatography. Subsequently, the resulting Compound X and 2-cyanoethyl-N,N,N′,N′-tetraisopropylphosphorodiamidite were reacted in acetonitrile in the presence of 1H-tetrazole on the basis of the technique described in The Journal of Organic Chemistry 62. 846-852 (1997) and Tetrahedron Letters 39. 9019-9022 (1998), so that an unrefined product of a phosphoamidite monomer XI(a), in which phosphoroamidide was added to the other hydroxyl group, was produced and, thereafter was separated and refined by the column chromatography.

A phosphoroamidite monomer XI(b) was synthesized in the same manner as that described above except that 4-phenylazobenzoic acid was used in place of 3-phenylazobenzoic acid VIII. Furthermore, a phosphoroamidite monomer XI(c) was synthesized in the same manner as that described above except that para-methyl red was used in place of 3-phenylazobenzoic acid VIII.

Finally, a chemically modified DNA enzyme including an azobenzene derivative, according to the present invention, was synthesized. In the present example, a 10-23 DNA enzyme was synthesized. For the synthesis of the chemically modified DNA enzyme, ABI394 type DNA synthesizer was used, phosphoamidite monomers XI(a) to XI(c) produced as described above and a commercially available phosphoamidite monomer corresponding to four natural bases were used, and DNA enzymes (DNA-1A: Sequence No. 4, DNA-1B: Sequence No. 5, and DNA-1C: Sequence No. 6) of the present invention having the following base sequences were synthesized. After unrefined products were produced on the basis of an usual protocol, the resulting unrefined products were refined by conducting gel refinement and high performance liquid chromatography refinement. For a comparative example, a DNA enzyme (DNA-N: Sequence No. 3) simply composed of four natural bases was synthesized in a manner similar to that described above. Each base sequence is shown in the following Table 1. In the base sequences, the underlined base sequences represent catalytically active loops.

TABLE 1

| | |
|---|---|
| DNA enzyme | Base sequence |

| | |
|---|---|
| DNA-N | 5′ - CTGAAGGGGGCTAGCTACAACGATTCTTCCT - 3′ |
| (Sequence No. 3) | |
| DNA-1A | 5′ - CTGAAGGGGGCTAGCTACAACGAX_{A}TTCTTCCT - 3′ |
| (Sequence No. 4) | |
| DNA-1B | 5′ - CTGAAGGGGGCTAGCTACAACGAX_{B}TTCTTCCT - 3′ |
| (Sequence No. 5) | |
| DNA-1C | 5′ - CTGAAGGGGGCTAGCTACAACGAX_{C}TTCTTCCT - 3′ |
| (Sequence No. 6) | |

Every DNA enzyme was identified on the basis of MALDI-TOFMS. The sequence of the RNA used as the substrate was as described below. In order to provide the substrate RNA with a fluorescence label, fluorescein isothiocyanate (FITC) represented by the following Formula:

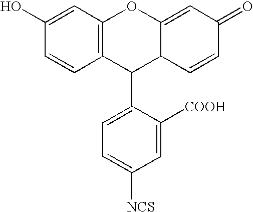

was introduced at a 5′ end.

| | |
|---|---|
| 5′-(FITC)-AGGAAGAAGCCCUUCAG-3′ | (Sequence No. 7) |

### Examples 1 to 3, Comparative Example 1

[RNA Cleavage Experiment]

The DNA enzymes (DNA-N: Sequence No. 3, DNA-1A: Sequence No. 4, DNA-1B: Sequence No. 5, and DNA-1C: Sequence No. 6) synthesized in Synthesis example 1 were used. The RNA cleavage experiment was conducted in accordance with the following procedure. First, 4 μL of DNA enzyme aqueous solution, 4 μL of substrate RNA aqueous solution, and furthermore, 4 μL of buffer aqueous solution were taken into a microtube, and agitation and mixing were conducted adequately at room temperature. The final concentration of each substance contained in the reaction solution was adjusted as described below.

| | | |
|---|---|---|
| DNA enzyme: | 16.0 | μmol/L |
| substrate RNA: | 1.6 | μmol/L |
| Tris-HCl: | 50 | mmol/L |
| magnesium chloride: | 10 | mmol/L |
| sodium chloride: | 1 | mol/L |

Next, the resulting reaction solution was transferred to a constant temperature bath adjusted at 37° C., and reaction was conducted for 1 hour with respect to Comparative example 1 (DNA-N: Sequence No. 3) and Examples 1 and 2 (DNA-1A: Sequence No. 4 and DNA-1B: Sequence No. 5), and for 40 minutes with respect to Example 3 (DNA-1C: Sequence No. 6). Thereafter, 12 μL of aqueous solution containing 10 mol/L of urea and 50 mmol/L of ethylenediaminetetraacetic acid was added to terminate the reaction, and cleavage pieces of the RNA and uncleaved RNA were separated by acrylamide gel electrophoresis. Finally, FITC in the resulting gel was excited by the light of 470 nm and the fluorescence intensity at 520 nm was monitored with a fluoroimager (FLA-3000: produced by Fuji Photo Film Co., Ltd.), so that the amount of cleavage of the RNA was quantified. The cleavage results are shown in the following Table 2.

TABLE 2

| | | Amount of |
|---|---|---|
| | DNA enzyme | cleavage (%) |
| Comparative | DNA-N | 12.5 |
| example 1 | (Sequence No. 3) | |
| Example 1 | DNA-1A | 38.8 |
| | (Sequence No. 4) | |
| Example 2 | DNA-1B | 36.0 |
| | (Sequence No. 5) | |
| Example 3 | DNA-1C | 33.3 |
| | (Sequence No. 6) | |

From the results shown in Table 2, it was ascertained that when a molecule having high planarity is chemically introduced at the 3′-side end of the catalytically active loop of the DNA enzyme, an RNA cleavage activity higher than that of the known DNA enzyme simply composed of natural bases is provided. Furthermore, since all three substances, meta-azo (Example 1), para-azo (Example 2), and methyl red (Example 3), have the same level of RNA cleavage activity, it is believed that if a substance is planar, intercalation and stabilization are possible.

### Examples 4 to 7, Comparative Example 2

“Optical Control of RNA Cleavage Activity”

DNA enzymes were additionally synthesized in accordance with the method in Synthesis example 1. The base sequencies thereof are shown in the following Table 3. In the base sequences, the underlined base sequences represent catalytically active loops.

TABLE 3

| | |
|---|---|
| DNA enzyme | Base sequence |

| | |
|---|---|
| DNA-2A | 5′ - CTGAAGGGGGCTAGCTACAACGATX_{A}TCTTCCT - 3′ |
| (Sequence No.8) | |
| DNA-3A | 5′ - CTGAAGGGGGCTAGCTACAACGATTCX_{A}TTCCT - 3′ |
| (Sequence No.9) | |

The resulting DNA enzyme was used, and a reaction solution was prepared at room temperature in the same manner as that in Examples 1 to 3. The reaction solution was transferred to a constant temperature bath at 37° C., and the reaction was conducted for a predetermined time while ultraviolet light was applied through a UV-D36C filter (produced by Asahi Techno Glass Corporation) by using a UV-A fluorescent lamp (FL6BL-A: produced by TOSHIBA CORPORATION). The intensity of the UV light under this condition was 100 μJ/cm² or less. In addition, a reaction solution having the same composition was reacted under the same condition except that no UV light was applied. Thereafter, as in Examples 1 to 3, an urea-EDTA solution was added to terminate the reaction, and cleavage pieces of the RNA and uncleaved RNA were separated by the acrylamide gel electrophoresis. Finally, a fluoroimager (FLA-3000: produced by Fuji Photo Film Co., Ltd.) was used, FITC in the resulting gel was excited by the light of 470 nm, and the fluorescence intensity at 520 nm was monitored, so that the amount of cleavage of the RNA was quantified. The cleavage results are shown in the following Table 4.

TABLE 4

| | | Amount of cleavage (%) | | |
|---|---|---|---|---|
| | | Under UV | | |
| | | light | No UV light | Reaction |
| | DNA enzyme | irradiation | irradiation | time |
| Comparative | DNA-N | 37.3 | 37.6 | 4 hours |
| example 2 | (Sequence No. 3) | | | |
| Example 4 | DNA-1A | 12.4 | 38.8 | 1 hour |
| | (Sequence No. 4) | | | |
| Example 5 | DNA-1B | 21.7 | 39.0 | 1 hour |
| | (Sequence No. 5) | | | |
| Example 6 | DNA-2A | 18.0 | 29.4 | 4 hours |
| | (Sequence No. 8) | | | |
| Example 7 | DNA-3A | 12.3 | 18.5 | 4 hours |
| | (Sequence No. 9) | | | |

From the results shown in Table 4, it was ascertained that when the light is applied at specific wavelengths to the DNA enzyme including a residue, to which an organic group is bonded, introduced at the 3′-side end of the catalytically active loop or in oligonucleotide complementary to the substrate RNA, wherein structural isomerization between a planar structure and a nonplanar structure of the organic group is effected by the light irradiation at the specific wavelengths, the reversible structural isomerization between a planar structure and a nonplanar structure of the organic group is effected by the light irradiation at the specific wavelengths and, thereby the RNA cleavage activity can be controlled. Likewise, it is believed that the RNA cleavage activity can be controlled by the light irradiation for a DNA enzyme including nucleotide residues, to which an organic group is bonded, introduced at the 3′-side end of the catalytically active loop and in oligonucleotide complementary to the substrate RNA, wherein structural isomerization between a planar structure and a nonplanar structure of the organic group is effected.

### INDUSTRIAL APPLICABILITY

When the high activity DNA enzyme according to the present invention is used, gene expression at a messenger RNA level can be suppressed more efficiently than ever. Furthermore, since the enzyme activity of a DNA enzyme can be controlled by the light irradiation, the gene expression can be reversibly controlled. Consequently, it is expected that the usefulness thereof is exhibited in various fields of biotechnology.

1. DNA enzyme characterized by comprising a nucleotide residue, to which any one organic group selected from the group consisting of azobenzene, spiropyran, stilbene, and derivatives thereof is bonded, at a 3′-side end of a catalytically active loop of the DNA enzyme.

2. The DNA enzyme according to claim 1, represented by the following Formula: 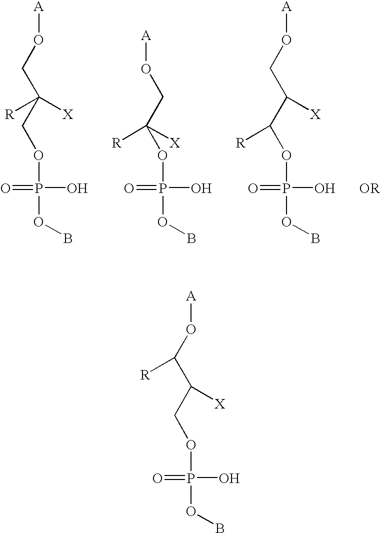
(in Formulae, A represents a catalytically active loop end, B represents nucleotide or oligonucleotide, X represents any one organic group selected from the group consisting of azobenzene, spiropyran, stilbene, and derivatives thereof, and R represents a hydrogen atom or an alkyl group having the carbon number of 1 to 4).

3. The DNA enzyme according to claim 2, wherein X is represented by the following Formula (I), (II), or (III): 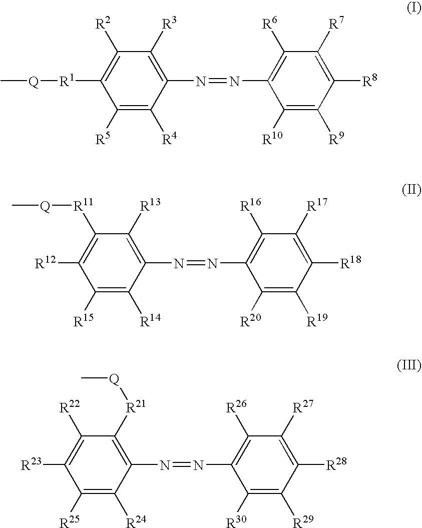
(in Formulae, R¹, R¹¹, and R²¹ represent independently a direct bond; an unsubstituted or a halogen atom-, hydroxyl-, amino-, nitro-, or carboxyl-substituted alkylene group having the carbon number of 1 to 20; or an unsubstituted or a halogen atom-, hydroxyl-, amino-, nitro-, or carboxyl-substituted alkenylene group having the carbon number of 2 to 20, Q represents a direct bond, an oxygen atom, a —(CH₂)ₙ—NH—CO— group, or a —(CH₂)ₙ—CO—NH— group, where n=1 to 5, and R² to R¹⁰, R¹² to R²⁰, and R²² to R³⁰ represent independently an unsubstituted or a halogen atom-, hydroxyl-, amino-, nitro-, or carboxyl-substituted alkyl group or alkoxy group having the carbon number of 1 to 20; an unsubstituted or a halogen atom-, hydroxyl-, amino-, nitro-, or carboxyl-substituted alkenyl group or alkynyl group having the carbon number of 2 to 20; a hydroxyl group; a halogen atom; an amino group; a nitro group; or a carboxyl group).

4. A method for controlling the activity of a DNA enzyme, characterized by comprising the step of applying light at specific wavelengths to the DNA enzyme including a nucleotide residue, to which any one organic group selected from the group consisting of azobenzene, spiropyran, stilbene, and derivatives thereof is bonded, and thereby, effecting reversible structural isomerization between a planar structure and a nonplanar structure of the organic group, so as to control the RNA cleavage activity of the DNA enzyme.

5. The method for controlling the activity of a DNA enzyme according to claim 4, wherein the introduction position of the nucleotide residue is a 3′-side end of a catalytically active loop.

6. The method for controlling the activity of a DNA enzyme according to claim 5, wherein the DNA enzyme is represented by the following Formula: 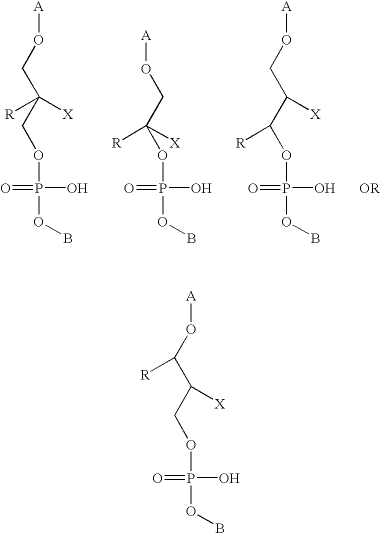
(in Formulae, A represents a catalytically active loop end, B represents nucleotide or oligonucleotide, X represents any one organic group selected from the group consisting of azobenzene, spiropyran, stilbene, and derivatives thereof, and R represents a hydrogen atom or an alkyl group having the carbon number of 1 to 4).

7. The method for controlling the activity of a DNA enzyme according to claim 6, wherein X is represented by the following Formula (IV), (V), or (VI): 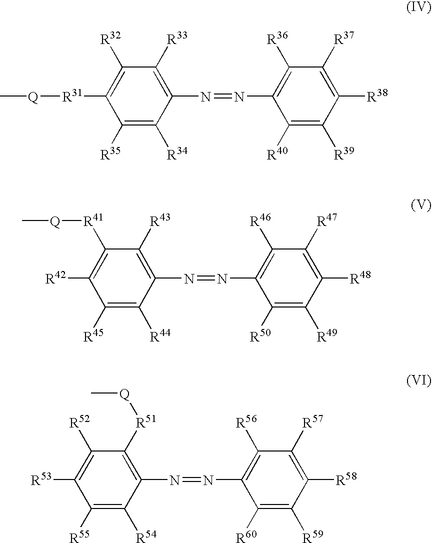
(in Formulae, R³¹, R⁴¹, and R⁵¹ represent independently a direct bond; an unsubstituted or a halogen atom-, hydroxyl-, amino-, nitro-, or carboxyl-substituted alkylene group having the carbon number of 1 to 20; or an unsubstituted or a halogen atom-, hydroxyl-, amino-, nitro-, or carboxyl-substituted alkenylene group having the carbon number of 2 to 20, Q represents a direct bond, an oxygen atom, a —(CH₂)ₙ—NH—CO— group, or a —(CH₂)ₙ—CO—NH— group, where n=1 to 5, R³² to R³⁷, R³⁹, R⁴⁰, R⁴² to R⁴⁷, R⁴⁹, R⁵⁰, R⁵² to R⁵⁷, R⁵⁹, and R⁶⁰ represent independently an unsubstituted or a halogen atom-, hydroxyl-, amino-, nitro-, or carboxyl-substituted alkyl group or alkoxy group having the carbon number of 1 to 20, an unsubstituted or a halogen atom-, hydroxyl-, amino-, nitro-, or carboxyl-substituted alkenyl group or alkynyl group having the carbon number of 2 to 20, a hydroxyl group; a halogen atom; an amino group; a nitro group; or a carboxyl group, and R³⁸, R⁴⁸, and R⁵⁸, represent independently an unsubstituted or a halogen atom-, hydroxyl-, amino-, nitro-, or carboxyl-substituted alkyl group or alkoxy group having the carbon number of 1 to 20; an unsubstituted or a halogen atom-, hydroxyl-, amino-, nitro-, or carboxyl-substituted alkenyl group or alkynyl group having the carbon number of 2 to 20; a hydroxyl group; or a halogen atom).
